(19) United States
(12) Patent Application Publication
(10) Pub. No.: US 2005/0181398 A1
(43) Pub. Date: Aug. 18, 2005

(54) Specific detection of host response protein clusters

(76) Inventors: Eric Thomas Fung, Mountain View, CA (US); Rebecca E. Caffrey, Houston, TX (US); Tai-Tung Yip, Cupertino, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 11/031,302

(22) Filed: Jan. 7, 2005

Related U.S. Application Data
(60) Provisional application No. 60/536,898, filed on Jan. 16, 2004.
(60) Provisional application No. 60/556,590, filed on Mar. 26, 2004.
(60) Provisional application No. 60/598,549, filed on Aug. 3, 2004.

(51) Int. Cl. C12Q 1/68; G06F 19/00; G01N 33/48; G01N 33/50

(52) U.S. Cl. 435/6; 702/20

(57) ABSTRACT

Methods of specifically detecting host response protein clusters and of correlating patterns of expression of these clusters with various clinical parameters are provided.

### CROSS-REFERENCE TO RELATED APPLICATIONS

This application claims the benefit of U.S. Provisional Applications Ser. Nos. 60/536,898, filed Jan. 16, 2004, 60/556,590, filed Mar. 26, 2004 and 60/598,549, filed Aug. 3, 2004, all of which applications are incorporated herein by reference in their entireties.

### FIELD

This invention relates to the fields of protein biochemistry and clinical diagnostics.

### BACKGROUND

Traditionally, diagnostic tests have focused on individual proteins whose relationship to the pathology could be clearly understood. For example, most traditional tumor markers are thought to have been shed by the cancer, either because cancer cells have entered the circulation or because cancer cells have been ingested by macrophages which in turn have entered the circulation and then been lysed, exposing tumor antigens. For the diagnosis of infectious disease, the typical diagnostic test is either a nucleic acid tests directed at DNA sequences specific to the infectious agent or an immunologic test directed at determining of the individual has produced antibodies specific to an antigen produced by the infectious agent. However, this paradigm for diagnostics has fallen short of its goal, particularly in cancer, cardiovascular, and neurologic testing. For example, prostate specific antigen is known to be elevated in conditions other than prostate cancer, including benign prostatic hyperplasia and in some breast cancers. CA125, a marker for ovarian cancer, is elevated in a number of other gynecologic conditions, both malignant and benign.

The ideal diagnostic test will have both high sensitivity and specificity, which can rarely be achieved using a single marker. This, in many ways, reflects the heterogeneity of human diseases, both in etiology and pathophysiology. For example, while “moderately-differentiated” colon cancer may have a common histologic appearance, there is abundant intratumoral and intertumoral molecular heterogeneity. Consequently it is not surprising that a single given molecular marker may be present in only a subset of cancers.

Because of the absence of highly accurate single markers for many diseases, attention has shifted to looking for an optimal combination of multiple markers. One approach is to make a priori assumptions regarding the relevance of several marker candidates and to determine if they, together, provide higher accuracy than they do individually. These are often called nomograms, of which the Partin table is an example for prostate cancer. A more powerful approach is to screen the combination of a large panel of candidate markers to find the optimal combination. Moreover, because proteins are post-translationally modified, a method that not only quantifies the candidate markers but determines the various post-translational modifications would be ideal.

The candidates that should be screened for their contribution to a potential multimarker diagnostic panel can come from multiple sources. As noted earlier, one approach is to extend the traditional paradigm. For example, the Partin table uses a combination of prostate specific antigen, clinical stage, and biopsy Gleason score to determine the likely pathologic stage. However, the traditional paradigm makes assumptions of questionable validity. A more general approach to identifying the candidates that should be screened is desirable.

It is well established that any disease leads to a host response, generally mediated by the innate immune system. This host response has generally been called the acute phase response and has a number of stereotyped constituents, broadly identified as positive acute phase reactants, which are up-regulated in disease, and negative acute phase reactants, which are down-regulated in disease. (Gebay, Cem and Kushner, Irving, Acute-phase proteins and other systemic responses to inflammation, New England Journal of Medicine, 1999, Vol. 340 (6), p. 448-454.) Most of the proteins that comprise the acute phase response are synthesized in the liver and secreted in the circulation. Moreover, many of these proteins have physiologic functions and are therefore expressed at some homeostatic level.

Thus, there is a need for more diagnostic tests and for improved tests that utilize multiple diagnostic markers.

### SUMMARY OF THE INVENTION

Methods are described here for discovering diagnostic patterns using host response protein clusters (discovery phase), and, second, methods of classifying or diagnosing a subject according to a disease based on the pattern of expression of host response proteins exhibited by the subject (clinical assay phase).

In one aspect, a method is described which comprises: (a) collecting samples from subjects belonging to at least two groups that differ according to a clinical parameter associated with disease; (b) measuring in each sample a plurality of host response protein clusters, wherein a cluster comprises a host response protein and at least one modified form of the host response protein; (c) submitting the measurements to a learning algorithm; and (d) generating a classification algorithm from the measurements that classifies a sample into at least one of the groups.

In a further aspect, the samples are selected from blood, urine, lymphatic fluid, cerebrospinal fluid, saliva, tears, milk, ductal lavage, semen, seminal plasma, vaginal secretions, tissue biopsy, cell extracts and cell culture supernatants and derivatives of these. In a further aspect, the clinical parameter is selected from presence or absence of disease, risk of disease, the stage of disease, response to treatment of disease and disease prognosis. In a further aspect, the disease is selected from an infectious disease, cancer, cardiovascular disease, autoimmune disease and prognosis. In a further aspect, the host response proteins are selected from C-reactive protein, transthyretin, apolipoprotein A1, apolipoprotein AII, apolipoprotein AIV, haptoglobin, interleukin 8, serum amyloid A (forms 1-4), inter-alpha trypsin inhibitor, complement factor, clotting cascade components, albumin, hemopexin, fetuin, transferrin, ceruloplasmin, serum proteases, and serum protease inhibitors and alpha-defensin.

In a further aspect, the method comprises measuring at least two different host response protein clusters selected from different classes of host response proteins, wherein the classes are selected from the group consisting of C reactive protein, transthyretin, apolipoprotein A1, apolipoprotein AII, apolipoprotein AIV, haptoglobin, interleukin 8, serum amyloid A (forms 1-4), inter-alpha trypsin inhibitor, complement factor, clotting cascade components, albumin, hemopexin, fetuin, transferrin, ceruloplasmin, serum proteases, and serum protease inhibitors and alpha-defensin.

In a further aspect, the method comprises measuring at least one positive acute phase protein cluster and at least one negative acute phase protein cluster. In a further aspect, the method comprises measuring in each sample at least four host response protein clusters.

In a further aspect the method comprises where at least one modified form is selected from a splice variant, RNA editing, or a post-translational modification, e.g. a product of enzymatic degradation, glycosylation, phosphorylation, lipidation, oxidation, methylation, cystinylation, sulphonation and acetylation.

In a further aspect, the method comprises wherein at least one modified form is selected from a product of enzymatic degradation, glycosylation, phosphorylation, lipidation, oxidation.

In a further aspect, the method further comprises measuring at least one protein that interacts with a protein from at least one cluster. In a further aspect, the method comprises at least one interactor protein that interacts with an antibody that binds to a host response protein, wherein the interactor protein is not the host response protein or a modified form thereof. In a further aspect, the measuring comprises capturing each host response protein cluster with at least one biospecific capture reagent that specifically recognizes the host response protein and measuring the captured proteins. In a further detailed aspect, the biospecific capture reagent is an antibody.

In a further aspect, the host response protein clusters are measured by mass spectrometry. In another aspect, the host response protein clusters are measured by affinity mass spectrometry.

In a further aspect, the learning algorithm is selected from linear regression processes, binary decision trees, artificial neural networks such as back-propagation networks, discriminant analyses, logistic classifiers, and support vector classifiers.

In a further aspect, the method comprises using the classification algorithm to classify an unknown sample from a test subject into one of the groups. In a further detailed aspect, the test subject presents a clinical parameter consistent with pathology. In another detailed aspect, the test subject does not present a clinical parameter consistent with pathology.

In another aspect, a method is described which comprises: (a) providing a learning set comprising a plurality of data objects representing subjects, wherein each data object comprises data representing measurements of a plurality of host response protein clusters from a subject sample, wherein each cluster comprises a host response protein and at least one modified form of the host response protein, and wherein the subjects are classified according to at least two different clinical parameters; and (b) training a learning algorithm with the learning set, thereby generating a classification model, wherein the classification model classifies a subject sample into a clinical parameter.

In a further aspect, the learning algorithm is unsupervised. In a further aspect, the learning algorithm is supervised and each data object further comprises data representing at least one clinical parameter of the subject. In some aspects, the supervised learning algorithm is selected from linear regression processes, binary decision trees, artificial neural networks, discriminant analyses, logistic classifiers, and support vector classifiers. In a detailed aspect, the supervised learning algorithm is a linear regression process selected from multiple linear regression (MLR), partial least squares (PLS) regression and principal components regression (PCR). In another detailed aspect, the supervised learning algorithm is a recursive partitioning processes. In further detailed aspect, the recursive partitioning processes is a classification and regression tree analysis.

In a further aspect, the supervised learning algorithm is a discriminant analysis selected from a Bayesian classifier or Fischer analysis.

In another aspect, the method further comprises: (1) submitting a data object to the classification algorithm for classification, wherein the data object represents a subject and comprises data representing measurements of proteins that are elements of the classification algorithm; and (2) using the classification algorithm to classify the subject. In a further aspect, the method is described which comprises measuring in a sample a plurality of host response protein clusters, wherein a cluster comprises a host response protein and at least one modified form of the host response protein.

In a further aspect, the method comprises measuring at least two different host response protein clusters selected from different classes of host response proteins, wherein the classes are selected from the group consisting of positive acute phase reactants and negative acute phase reactants. In a further aspect, the clusters are selected from C reactive protein, transthyretin, apolipoprotein A1, apolipoprotein AII, apolipoprotein AIV, haptoglobin, interleukin 8, serum amyloid A (forms 1-4), inter-alpha trypsin inhibitor, complement factor, components of the clotting cascade, albumin, hemopexin, fetuin, transferring, ceruloplasmin, serum proteases and serum protease inhibitors, and alpha-defensin.

In a further aspect, the proteins clusters are measured by mass spectrometry. In another aspect the proteins clusters are measured by affinity mass spectrometry. In a further detailed aspect, affinity mass spectrometry further comprises SEND. In further aspect, the measuring comprises capturing each host response protein cluster with at least one biospecific capture reagent that specifically recognizes the host response protein and measuring the captured proteins.

In another aspect, a method is described which comprises: (a) measuring a plurality of proteins in a sample, wherein the proteins are selected from host response proteins, modified forms of host response proteins and protein interactors with these, wherein the proteins are elements of a classification algorithm that classifies a sample into a group based on a clinical parameter, wherein the classification algorithm is generated according to the method of claim 21. In another aspect, the method further comprises (b) using the classification algorithm to classify the sample into a group based on the clinical parameter.

In another aspect, a kit is described comprising: (a) a plurality of biospecific capture reagents, wherein each capture reagent is attached to a different solid support or to a different addressable location on the same solid support or a combination of these, and wherein at least two of the capture reagents specifically bind to different host response protein clusters In a further aspect, the solid support is a mass spectrometer probe.

In another aspect, a kit is described comprising a plurality of containers, each container comprising a different biospecific capture reagent, wherein each capture reagent specifically binds to a different host response protein cluster. In a further aspect at least one solid support comprises a reactive functionality for coupling a biospecific capture reagent to the solid support. In a further aspect, the different host response proteins are selected from different classes, wherein the classes are selected from positive acute phase reactants and negative acute phase reactants.

In another aspect, a method is described which comprises measuring a clinical parameter in a subject. The method comprises measuring in a sample from the subject a plurality of host response protein clusters and correlating the measurement with a clinical parameter. In a further aspect, the clinical parameter is selected from presence or absence of disease, risk of disease, the stage of disease, response to treatment of disease and disease prognosis.

In another aspect, a method for assessing the presence or absence of a disease state in a subject is described. The method comprises measuring in a sample from the subject a plurality of host response protein clusters and correlating the measurement with the presence or absence of the disease state.

In another aspect, a method is described which comprises: (a) collecting samples from subjects belonging to at least two groups that differ according to a clinical parameter associated with disease; (b) measuring in each sample a plurality of host response proteins; (c) submitting the measurements to a learning algorithm; and (d) generating a classification algorithm from the measurements that classifies a sample into at least one of the groups. In a further aspect, at least 4, at least 10 at least 25, at least 50 or at least 100 different host response proteins are measured.

In another aspect, a method is described which comprises: (a) measuring a plurality of host response proteins in a sample, wherein the proteins are elements of a classification algorithm that classifies a sample into a group based on a clinical parameter; and (b) using the classification algorithm to classify the sample into a group characterized by clinical parameter.

### BRIEF DESCRIPTION OF THE DRAWINGS

FIG. 1 (Sections 1.1-1.3) shows a protocol for the discovery phase of identifying host response protein markers that are diagnostic for a particular clinical parameter.

FIG. 2 (Sections 2.1-2.4) shows a protocol for the assay phase of using the discovered markers to diagnose a subject.

### DETAILED DESCRIPTION

I. Introduction

It is known that the body expresses any of a number of proteins, referred to as “host response proteins” in response to a variety of pathological states, such as infection or cancer. The inventors have discovered that the pattern of expression of host response proteins is characteristic of particular pathological conditions. That is to say, different diseases and/or inciting events (e.g., inflammation, cancer, infection, and the like) elicit different individual components of the acute phase response and accordingly the relative level of expression of these individual components, e.g., host response proteins, characterizes the disease state or inciting event. Therefore, the pattern of expression of these proteins that characterizes a particular disease can be discovered, and the pattern can be used to determine whether a subject has the particular disease. Furthermore, the ability to diagnose or classify is significantly improved when host response proteins are measured as a cluster, that is, the intact protein as well as the modified forms of the intact protein found in a subject sample. Quantifying individual forms of hosts response protein instead of total host response protein can confer higher specificity and thus enables a clinician to more accurately classify a sample as belonging to a specific clinical parameter associated with a disease state. This is particularly true when measuring relatively abundant host response proteins that respond to many inciting events that occur within the body, including for example, inflammation, infection, vascular disease, and malignancy. This discriminatory ability can be further improved by also measuring proteins that interact with one or more proteins in the host response protein cluster.

Accordingly, this invention provides, first, methods discovering diagnostic patterns using host response protein clusters (discovery phase), and, second, methods of classifying or diagnosing a subject according to a disease based on the pattern of expression of host response proteins exhibited by the subject (clinical assay phase). As both methods involve the specific detection of host response proteins, a discussion of host response proteins and methods of specifically detecting host response protein clusters is now appropriate.

II. Host Response Proteins

The host response comprises a cascade of inflammatory signals that can be triggered by very small inciting events and that leads to up- and down- regulation of a group of circulating proteins called host response proteins. Host response proteins are generally described as positive acute phase reactants and negative acute phase reactants. An acute phase reactant, also known as an acute phase protein, is a protein whose plasma concentration increases by at least about 25% during inflammatory disorders. Conversely, a negative acute phase reactant or negative acute phase protein is one whose plasma concentration decreases by at least about 25% during inflammatory disorders. Specific classes of positive acute phase reactants include complement factors such as C2, C3, C4, C8, C9, Factor B, Factor H, C1 inhibitor, C4b-binding protein, and mannose-binding lectin; clotting factors such as fibrinogen, plasminogen, tissue plasminogen activator, urokinase, Protein S, vitronectin, and plasminogen activator inhibitor-1; serum proteases and protease inhibitors such as α₁-protease inhibitor, α₁-antichymotrypsin, α₁-antitrypsin, inter-α trypsin inhibitor heavy chain four, pancreatic secretory trypsin inhibitor, and inter-α-trypsin inhibitors; transport proteins such as haptoglobin, hemopexin, and ceruloplasmin; inflammatory mediators such as secreted phospholipase A₂, lipopolysaccharide-binding protein, interleukin-1-receptor antagonist, and granulocyte colony-stimulating factor; and other proteins such as serum amyloid A, C-reactive protein, lipoprotein A, apolipoprotein A1, apolipoprotein B, α₁-acid glycoprotein, fibronectin, ferritin, α₂-macroglobulin, ceruloplasm, and angiotensinogen. Specific examples of negative acute phase reactants include albumin, transthyretin, transferrin, fetuin, insulin-like growth factor, α₂-HS glycoprotein, alpha-fetoprotein, thyroxine-binding globulin, and factor XII.

In some embodiments, the host response proteins are selected from C-reactive protein, transthyretin, apolipoprotein A1, apolipoprotein AII, apolipoprotein AIV, haptoglobin, interleukin 8, serum amyloid A (forms 1-4), inter-alpha trypsin inhibitor, complement factor, clotting cascade components, albumin, hemopexin, fetuin, transferrin, ceruloplasmin, serum proteases, and serum protease inhibitors and alpha-defensin.

Host response proteins, like other proteins, can exist in a sample in many different forms. These include both pre- and post-translationally modified forms. Pre-translational modified forms include allelic variants, slice variants and RNA editing forms. Post-translationally modified forms include forms resulting from proteolytic cleavage (e.g., fragments of a parent protein), glycosylation, phosphorylation, lipidation, oxidation, methylation, cystinylation, sulphonation and acetylation.

In a preferred embodiment, the host response protein clusters represent a subset of host response proteins that are differentially expressed in response to different inciting events and disease states.

III. Specific Detection of Host Response Protein Clusters and Biomolecular Interactors

Both the discovery phase and the assay phase involve the specific detection and measurement of a host response protein, modified forms of it and biomolecular interactors with these. Measuring a protein or its modified forms can involve detecting the presence or absence of the protein, in a sample or quantifying the amount in relative or absolute terms. A relative amount could be, for example, high, medium or low. An absolute amount could reflect the measured strength of a signal or the translation of this signal strength into another quantitative format, such as micrograms/ml.

The polypeptides of this invention can be detected by any suitable method. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

However, in preferred embodiments the detection strategy involves first capturing the host response proteins and their interactors and then detecting by mass spectrometry. More specifically, the proteins are captured using biospecific capture reagents, such as antibodies, that recognize a host cell protein and modified forms of it. This will also result in the capture of protein interactors that are bound to the host response proteins or that are otherwise recognized by antibodies. Preferably, the biospecific capture reagents are bound to a solid phase. Then, the captured proteins can be detected by SELDI mass spectrometry or by eluting the proteins from the capture reagent and detecting the eluted proteins by traditional MALDI or by SELDI. The use of mass spectrometry is especially attractive because it can distinguish and quantitate modified forms of a protein based on mass and without the need for labeling.

A. CAPTURE WITH BIOSPECIFIC CAPTURE REAGENTS

In one embodiment, each host response protein cluster and biomolecular interactors of them are captured with biospecific capture reagents. Biospecific adsorbents include those molecules that bind a target analyte with an affinity of at least 10⁻⁹ M, 10⁻¹⁰ M, 10⁻¹¹ M or 10⁻¹² M. Many biospecific capture reagents are known in the art including, for example, antibodies, binding fragments of antibodies (e.g., single chain antibodies, Fab′ fragments, F(ab)′2 fragments, and scFv proteins), affibodies (Affibody, Teknikringen 30, floor 6, Box 700 04, Stockholm SE-10044, Sweden, U.S. Pat. No.: 5,831,012)) and nucleic acid protein fusions (e.g., from Phylos, Lexington, Mass.). Depending on intended use, they also may include receptors and other proteins that specifically bind another biomolecule.

More particularly, the inventors recognize that a biospecific capture reagent, such as an antibody, directed against a particular host response protein will capture modified forms of the host response protein, in particular, fragments, that comprise the epitope recognized by the antibody. In fact, by utilizing biospecific capture reagents that recognize different epitopes on the same host response protein, one can capture modified forms with one antibody that another antibody may not recognize.

Furthermore, the biospecific capture reagent will also capture proteins that interact with, and are bound to, the proteins directly recognized by the biospecific capture reagent. Proteins and the proteins that interact with them are referred to as the “interactome.” In a sample, a host response protein may be bound to other proteins that interact with it. A biospecific capture reagent that captures the host response protein or its modified forms also will capture any proteins that interact with them. Recovery of these interacting proteins will depend upon the stringency with which the antibody-protein complex is treated. Furthermore, an antibody also may capture proteins other than the host response protein or modified forms to which it is directed that also comprise the target epitope. One can then choose a washing condition is has sufficient stringency to remove proteins that are unbound or that bind non-specifically, but not so stringent as to remove these interacting proteins. In this way, one can capture the target protein, its modified forms and proteins that interact with either.

Preferably, the biospecific capture reagent is bound to a solid phase, such as a bead, a plate or a chip. Methods of coupling biomolecules, such as antibodies, to a solid phase are well known in the art. They can employ, for example, bifunctional linking agents, or the solid phase can be derivatized with a reactive group, such as an epoxide or an imidizole, that will bind the molecule on contact. Biospecific capture reagents against different target host response proteins can be mixed in the same place, or they can be attached to solid phases in different physical or addressable locations. For example, one can load multiple columns with derivatized beads, each column able to capture a single host response protein cluster. Alternatively, one can pack a single column with different beads derivatized with capture reagents against a variety of host response protein clusters, thereby capturing all the analytes in a single place. Accordingly, antibody-derivatized bead-based technologies, such as xMAP technology of Luminex (Austin, Tex.) can be used to detect the host response protein clusters. However, the biospecific capture reagents must be specifically directed toward the members of a cluster in order to differentiate them.

In yet another embodiment, the surfaces of biochips can be derivatized with the capture reagents directed against host response protein clusters either in the same location or in physically different addressable locations. One advantage of capturing different clusters in different addressable locations is that the analysis becomes simpler.

In another embodiment, host response protein, modified forms of host response protein or biomolecular interactors of these can be measured by immunoassay. Immunoassay requires biospecific capture reagents, such as antibodies, to capture the analytes. Furthermore, the assay can be designed to specifically distinguish host response protein and modified forms of host response protein. This can be done, for example, by employing a sandwich assay in which one antibody captures more than one form and second, distinctly labeled antibodies, specifically bind, and provide distinct detection of, the various forms. Antibodies can be produced by immunizing animals with the biomolecules. This invention contemplates traditional immunoassays including, for example, sandwich immunoassays including ELISA or fluorescence-based immunoassays, as well as other enzyme immunoassays.

B. DETECTION BY MASS SPECTROMETRY

In a preferred embodiment, host response proteins are detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these.

In a further preferred method, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer.

1. SELDI

A preferred mass spectrometric technique for use in the invention is “Surface Enhanced Laser Desorption and Ionization” or “SELDI,” as described, for example, in U.S. Pat. Nos. 5,719,060 and 6,225,047, both to Hutchens and Yip. This refers to a method of desorption/ionization gas phase ion spectrometry (e.g., mass spectrometry) in which an analyte (here, one or more of the host response proteins) is captured on the surface of a SELDI mass spectrometry probe. There are several versions of SELDI.

One version of SELDI is called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent may be attached directly to the substrate of the selective surface, or the substrate may have a reactive surface that carries a reactive moiety that is capable of binding the capture reagent, e.g., through a reaction forming a covalent or coordinate covalent bond. Epoxide and acyl-imidizole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

Chromatographic adsorbents include those adsorbent materials typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

Biospecific adsorbents include those molecules that specifically bind to a biomolecule. Typically they comprise a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸ M.

Protein biochips produced by Ciphergen Biosystems, Inc. comprise surfaces having chromatographic or biospecific adsorbents attached thereto at addressable locations. Ciphergen ProteinChip® arrays include NP20 (hydrophilic); H4 and H50 (hydrophobic); SAX-2, Q-10 and LSAX-30 (anion exchange); WCX-2, CM-10 and LWCX-30 (cation exchange); IMAC-3, IMAC-30 and IMAC 40 (metal chelate); and PS-10, PS-20 (reactive surface with acyl-imidizole, expoxide) and PG-20 (protein G coupled through acyl-imidizole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have nitrilotriacetic acid functionalities that adsorb transition metal ions, such as copper, nickel, zinc, and gallium, by chelation. Preactivated ProteinChip arrays have acyl-imidizole or epoxide functional groups that can react with groups on proteins for covalent binding.

Such biochips are further described in: U.S. Pat. No. 6,579,719, Hutchens and Yip, Jun. 17, 2003; PCT Publication No. WO 00/66265 Rich et al., Nov. 9, 2000; U.S. Pat. No. 6,555,813, Beecher et al., Apr. 29, 2003; U.S. Pat. Application No. U.S. 2003 0032043 A1, Pohl and Papanu, Jul. 16, 2002; and PCT Publication No. WO 03/040700, Um et al., “Hydrophobic Surface Chip,” May 15, 2003); U.S. Provisional Pat. Application No. 60/367,837 Boschetti et al.,” May 5, 2002; and U.S. Pat. Application No. 60/448,467, Huang et al., filed Feb. 21, 2003.

In general, a probe with an adsorbent surface is contacted with the sample for a period of time sufficient to allow proteins that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound proteins.

The proteins bound to the substrates are detected in a gas phase ion spectrometer such as a time-of-flight mass spectrometer. The proteins are ionized by an ionization source such as a laser, the generated ions are collected by an ion optic assembly, and then a mass analyzer disperses and analyzes the passing ions. The detector then translates information of the detected ions into mass-to-charge ratios. Detection of a protein typically will involve detection of signal intensity. Thus, both the quantity and mass of the protein can be determined.

Another version of SELDI is Surface-Enhanced Neat Desorption (SEND), which involves the use of probes comprising energy absorbing molecules that are chemically bound to the probe surface (“SEND probe”). The phrase “energy absorbing molecules” (EAM) denotes molecules that are capable of absorbing energy from a laser desorption/ionization source and, thereafter, contribute to desorption and ionization of analyte molecules in contact therewith. The EAM category includes molecules used in MALDI, frequently referred to as “matrix,” and is exemplified by cinnamic acid derivatives, sinapinic acid (SPA), cyano-hydroxy-cinnamic acid (CHCA) and dihydroxybenzoic acid, ferulic acid, and hydroxyaceto-phenone derivatives. In certain embodiments, the energy absorbing molecule is incorporated into a linear or cross-linked polymer, e.g., a polymethacrylate. For example, the composition can be a co-polymer of α-cyano-4-methacryloyloxycinnamic acid and acrylate. In another embodiment, the composition is a co-polymer of α-cyano-4-methacryloyloxycinnamic acid, acrylate and 3-(tri-ethoxy)silyl propyl methacrylate. In another embodiment, the composition is a co-polymer of α-cyano-4-methacryloyloxycinnamic acid and octadecylmethacrylate (“C18 SEND”). SEND is further described in U.S. Pat. No. 6,124,137 and PCT Publication No. WO 03/64594, Kitagawa, Aug. 7, 2003.

SEAC/SEND is a version of SELDI in which both a capture reagent and an energy absorbing molecule are attached to the sample presenting surface. SEAC/SEND probes therefore allow the capture of analytes through affinity capture and ionization/desorption without the need to apply external matrix. The C18 SEND biochip is a version of SEAC/SEND, comprising a C18 moiety which functions as a capture reagent, and a CHCA moiety which functions as an energy absorbing moiety.

Another version of SELDI, called Surface-Enhanced Photolabile Attachment and Release (SEPAR), involves the use of probes having moieties attached to the surface that can covalently bind an analyte, and then release the analyte through breaking a photolabile bond in the moiety after exposure to light, e.g., to laser light, see, U.S. Pat. No. 5,719,060. SEPAR and other forms of SELDI are readily adapted to detecting a protein or protein profile, pursuant to the present invention.

2. Other Mass Spectrometry Methods

In another mass spectrometry method, the proteins can be first captured on a chromatographic resin that binds the target molecules. For example, the resin can be derivatized with anti-host response proteins antibodies. Alternatively, this method could be preceded by chromatographic fractionation before application to the bio-affinity resin. After elution from the resin, the sample can be analyzed by MALDI, electrospray, or another ionization method for mass spectrometry. In another alternative, one could fractionate on an anion exchange resin and detect by MALDI or electrospray mass spectrometry directly. In yet another method, one could capture the proteins on an immuno-chromatographic resin that comprises antibodies that bind the proteins, wash the resin to remove unbound material, elute the proteins from the resin and detect the eluted proteins by MALDI, SELDI, electrospray mass spectrometry or another ionization mass spectrometry method.

3. Data Analysis

Analysis of analytes by time-of-flight mass spectrometry generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation to generate a mass spectrum, baseline subtraction to eliminate instrument offsets and high frequency noise filtering to reduce high frequency noise.

Data generated by desorption and detection of proteins can be analyzed with the use of a programmable digital computer. The computer program analyzes the data to indicate the number of proteins detected, and optionally the strength of the signal and the determined molecular mass for each protein detected. Data analysis can include steps of determining signal strength of a protein and removing data deviating from a predetermined statistical distribution. For example, the observed peaks can be normalized, by calculating the height of each peak relative to some reference. The reference can be background noise generated by the instrument and chemicals such as the energy absorbing molecule which is set at zero in the scale.

The computer can transform the resulting data into various formats for display. The standard spectrum can be displayed, but in one useful format only the peak height and mass information are retained from the spectrum view, yielding a cleaner image and enabling proteins with nearly identical molecular weights to be more easily seen. In another useful format, two or more spectra are compared, conveniently highlighting unique proteins and proteins that are up- or down-regulated between samples. Using any of these formats, one can readily determine whether a particular protein is present in a sample.

Analysis generally involves the identification of peaks in the spectrum that represent signal from an analyte. Peak selection can be done visually, but software is available, as part of Ciphergen's ProteinChip® software package, that can automate the detection of peaks. In general, this software functions by identifying signals having a signal-to-noise ratio above a selected threshold and labeling the mass of the peak at the centroid of the peak signal. In one useful application, many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Software used to analyze the data can include code that applies an algorithm to the analysis of the signal to determine whether the signal represents a peak in a signal that corresponds to a protein according to the present invention. The software also can subject the data regarding observed protein peaks to classification tree or ANN analysis, to determine whether a protein peak or combination of protein peaks is present that indicates the status of the particular clinical parameter under examination. Analysis of the data may be “keyed” to a variety of parameters that are obtained, either directly or indirectly, from the mass spectrometric analysis of the sample. These parameters include, but are not limited to, the presence or absence of one or more peaks, the shape of a peak or group of peaks, the height of one or more peaks, the log of the height of one or more peaks, and other arithmetic manipulations of peak height data.

C. DETECTION BY IMMUNOASSAY

In another embodiment, the host response proteins can be measured by immunoassay. Immunoassay requires biospecific capture reagents, such as antibodies, to capture the proteins. Antibodies can be produced by methods well known in the art, e.g., by immunizing animals with the proteins. Proteins can be isolated from samples based on their binding characteristics. Alternatively, if the amino acid sequence of a host response protein is known, the polypeptide can be synthesized and used to generate antibodies by methods well known in the art.

This invention contemplates traditional immunoassays including, for example, sandwich immunoassays including ELISA or fluorescence-based immunoassays, as well as other enzyme immunoassays. In the SELDI-based immunoassay, a biospecific capture reagent for the protein is attached to the surface of an MS probe, such as a pre-activated ProteinChip array. The protein is then specifically captured on the biochip through this reagent, and the captured protein is detected by mass spectrometry.

Biospecific adsorbents include those molecules that bind a target analyte with an affinity of at least 10⁻⁹ M, 10⁻¹⁰ M, 10⁻¹¹ M or 10⁻¹² M. As is well understood in the art, biospecific capture reagents include antibodies, binding fragments of antibodies (e.g., single chain antibodies, Fab′ fragments, F(ab)′2 fragments, and scFv proteins and antibodies (Affibody, Teknikringen 30, floor 6, Box 700 04, Stockholm SE-10044, Sweden, U.S. Pat. No: 5,831,012). Depending on intended use, they also may include receptors and other proteins that specifically bind another biomolecule.

IV. DISCOVERY PHASE

The discovery of protein patterns from host response protein clusters involves four steps: (1) Collecting samples for analysis from subjects belonging to two or more groups to be compared; (2) measuring a plurality of host response protein clusters from the samples; (3) subjecting the resulting measurements to pattern analysis, for example submitting the data to learning algorithm and (4) generating a classification pattern, e.g., a classification algorithm, from the data that can classify a sample into one of the original groups.

A. COLLECTING SAMPLES

The discovery phase involves collecting samples from subjects that fall into at least two groups, based on a particular clinical parameter of interest. Typically, the subjects will fall into two groups: One group characterized by a clinical parameter of interest, and the other group characterized by not having the clinical parameter. Most typically the groups will be disease versus non-disease. However, it also may be useful to distinguish between two or more stages of a disease or between two or more different diseases. Diseases of interest include, for example, cancer, infectious disease (e.g., bacterial infection, viral infection, parasitic infection), cardiovascular disease (e.g., occurrence of myocardial infarction, degree of congestive heart failure), autoimmune disease and neurological disease (e.g, Alzheimer's disease, schizophrenia). It also may be useful to distinguish between two or more prognoses for a disease. It also may be useful to distinguish between two or more types of responses to therapy (e.g., responders v. non-responders) or two or more types of toxic responses to compound exposure (e.g., toxic response to compound v. non-toxic response to compound).

Generally, the greater the number of samples from each group, the more confidence one can have that the ultimate pattern generated can correctly classify a sample from the testable population. Thus for example, the number of samples from each group could be at least 10, at least 100 or at least 1000.

The samples can be of any biological material that appears relevant to the diagnostician as a material for clinical diagnosis. For example, the material can be selected from human and animal body fluid such as whole blood, plasma, white blood cells, cerebrospinal fluid, urine, semen, vaginal secretions, lymphatic fluid, and various external secretions of the respiratory, intestinal and genitourinary tracts, tears, saliva, milk, ductal lavage, seminal plasma, tissue biopsy, fixed tissue specimens, fixed cell specimens, cell extracts and cell culture supernatents and derivatives of these, e.g., blood or a blood derivative such as serum.

The samples may be subject to pre-processing before analysis. For example, blood may be fractionated into serum or plasma. Samples may be separated into different fractions by chromatography. Fractionation of a sample may be useful to simplify the sample for further analysis.

B. MEASURING HOST RESPONSE PROTEINS

Then, each sample is analyzed to detect the expression of a plurality of different host response protein clusters and/or interacting proteins. As stated, a host response protein cluster comprises a target protein and various modified forms of the protein, such as fragments. Generally, the proteins in a cluster will be recognized by one or more antibodies directed at one or more epitopes of the parent protein, insofar as the modified forms also comprise the target epitope. Similarly, an interacting protein can be captured and detected by capturing the protein to which it interacts.

The number of host response protein clusters must be at least two, but preferably includes many different host response proteins, as this provides more data in which to discover a diagnostic pattern. Thus, the number of host response protein clusters measured can be at least 2, at least 4, at least 8, at least 16, at least 32, at least 64 or at least 128. In one embodiment, the different host response protein clusters can be selected from within a single group of host response proteins. For example, one can measure a plurality of interleukins, or a plurality of cytokines, and the like. In another embodiment, the plurality of host response protein clusters comprises at least two host response protein clusters selected from at least two different classes of host response proteins, wherein the classes are selected from the group consisting of positive acute phase reactants and negative acute phase reactants. Specific classes of positive acute phase reactants include most complement factors, most clotting factors, serum proteases and protease inhibitors, transport proteins such as haptoglobin and hemopexin, and inflammatory mediators such as serum amyloid A, c-reactive protein. Specific examples of negative acute phase reactants include albumin, transthyretin, transferring, fetuin, and insulin-like growth factor. For example, the plurality can comprise at least one interleukin, at least one cytokine, at least one chemokine, etc. In certain embodiments, the plurality will include a plurality of different host response proteins from a plurality of different classes.

The value of measuring a plurality of clusters in different classes lies in the generation of a large amount of data from which subtle patterns can be discerned. The pattern that eventually emerges probably will not use all the proteins measured, but is likely to be more accurate than a pattern detected from only a few data points.

The assays just described produce a data set that represent several levels of analysis: (1) The detection of a plurality of forms of a host response protein and interactors (a host response protein cluster); (2) the detection of clusters for a plurality of different host response proteins; (3) the detection of different protein clusters in a plurality of samples classed into at least two different clinical groups (e.g., disease v. non-disease); and (4) the detection of different protein cluster in a plurality of samples classed into multiple clinical groups (e.g., disease A v. disease B v. disease C). Analysis of this data set provides the expression patterns that can be used to classify a sample into one of the clinical groups.

C. PATTERN ANALYSIS

Data generated from the measurement of host response protein clusters from the subject samples is then submitted for pattern recognition. While one can identify patterns by visual inspection of the data, in the case of large amounts of data it is preferred to subject the data to a learning algorithm executed by a computer. In this case, pattern analysis involves training a leaming algorithm with a leaming set of data that includes measurements of the aforementioned molecules and generating a classification algorithm that can classify an unknown sample into a class represented by clinical parameter.

The method involves, first, providing a learning set of data. The learning set includes data objects. Each data object represents a subject for which measurements have been made. The data included in the data object includes the specific measurements of host response protein, modified forms of host response protein and biomolecular interactors with these. Each subject is classified into one of the different clinical parameter classes under analysis, for example, presence or absence of disease, risk of disease, stage of disease, response to treatment of disease or class, prognosis, or kind of disease.

In a preferred embodiment, the learning set will be in the form of a table in which, for example, each row is data object representing a sample. The columns can contain information identifying the subject, data providing the specific measurements of each of the molecules measured and optionally identifying the clinical parameter associated with the subject.

The learning set is then used to train a classification algorithm. Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, IEEE Transactions on Pattern Analysis and Machine Intelligence, 22:1, 2000.

In supervised classification, each data object includes data indicating the clinical parameter class to which the subject belongs. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines). A preferred supervised classification method is a recursive partitioning process. Recursive partitioning processes use recursive partitioning trees to classify spectra derived from unknown samples.

In other embodiments, the classification models that are created can be formed using unsupervised learning methods. Unsupervised classification attempts to learn classifications based on similarities in the training data set. In this case, the data representing the class to which the subject belongs is not included in the data object representing that subject, or such data is not used in the analysis. Unsupervised learning methods include cluster analyses. Clustering techniques include the MacQueen's K-means algorithm and the Kohonen's Self-Organizing Map algorithm.

Learning algorithms asserted for use in classifying biological information are described, for example, in PCT Publication No. WO 01/31580, Barnhill et al.; U.S. Pat. Application 2002 0193950 A1, Gavin et al.; U.S. Pat. Application 2003 0004402 A1, Hitt et al.; and U.S. Pat. Application 2003 0055615 A1, Zhang and Zhang.

D. CLASSIFICATION PATTERN

Thus trained, learning algorithm will generate a classification model or algorithm that classifies a sample into one of the classification groups. The classification model usually involves a subset of all the markers included in the learning set. The classification model can be used to classify an unknown sample into one of the groups.

A learning algorithm, such as CART, can detect many different patterns in the learning set that are useful for classifying a sample into one of the groups. These patterns most likely will differ based not only on the specific markers employed in the classification algorithm, but also in the specific function of amount of the molecule in the sample (e.g., the cut-off value). However, it also is typical that among many patterns generated, certain of the proteins recur frequently, indicating that they are particularly useful as “splitters” in classification algorithms to classify a sample into one group or another.

V. CLINICAL ASSAY PHASE

Once the learning algorithm has generated a classification algorithm, the classification algorithm can be used in a clinical setting to classify a subject sample according to the clinical parameter that is the subject of the test. The clinical assay phase can include one or more of the following steps: (1) collecting a sample from a subject to be tested; (2) measuring the particular analytes from among the host response protein clusters or interactors that form the classification pattern; (3) comparing this data to the diagnostic classification pattern; e.g., submitting the data to the classification algorithm and (4) assigning the sample to one of the groups based on the pattern, e.g., based on the result of application of the classification algorithm.

This method involves measuring a plurality of biomarkers, e.g., proteins, in a sample from a subject. The selected biomarkers will be those that have been shown to have power in discriminating the various clinical parameters of interest, e.g., disease versus non-disease, stage of disease, propensity to develop disease, ability to respond to a treatment, etc. The collection of measurements represents a biomarker profile for the subject. This profile is then subjected to analysis to classify the sample, e.g., to form a diagnosis. The analysis can involve comparison with a reference profile that represents one of the states. However, while such a comparison is simple in the case of a single biomarker, it can be very difficult in the case of a plurality of biomarkers. In that case, the sample profile can be subject to a computer algorithm, e.g., a classification algorithm that performs a calculation reliably determining what state the subject is in.

The classification algorithm is keyed to the particular assay conditions under which it was developed. That is to say, in order to generate a useful result from a clinical test, it must be performed according to the same protocol as used to generate the data which was submitted to the learning algorithm. Changes in parameters such as sample source and measurement assay conditions will most likely result in data that cannot be properly interpreted by the classification algorithm. This is because the classification algorithm is likely to key on subtle relationships between particular molecules (the “pattern”). These relationships will probably be disrupted if different clinical assay conditions are used. For example, the use of a different wash buffer on a chip might alter the relative amount of two proteins retained on the chip. If this relative amount is used in the classification algorithm, then changing it by changing the assay conditions will also change the result of the test.

As stated, the proteins used in the classification algorithm will generally be a subset of the host response protein clusters measured in the discovery phase. Accordingly, in carrying out a clinical diagnostic assay keyed to the proteins in the classification algorithm, one need only specifically measure those host response proteins. These measurements then can be submitted to the classification algorithm for analysis. Alternatively, measurements can be obtained for a broad spectrum of host response proteins. Absence of changes for subsets of these proteins can, in fact, contribute to the specificity of the diagnosis.

Upon submission of the specific measurements called for the classification algorithm, the algorithm will generate a classification of the sample into one of the clinical parameters to which the test is directed. This result can aid the diagnostician by indicating that a particular clinical parameter is present, or by ruling out certain clinical parameters.

One can then manage subject treatment based on the result of the diagnostic test. For example, if disease is present, a certain course of treatment can be prescribed. Alternatively, if the result is ambiguous, further texts can be ordered. Tests can be performed sequentially, to provide monitoring of a patient for the progression of the disease or the effect of treatment or the status of recovery.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of actual positives that test as positive. Negative predictive value is the percentage of actual negatives that test as negative.

VI. KITS FOR DETECTION OF HOST RESPONSE PROTEIN CLUSTERS

In another aspect, the present invention provides kits for discovering or assaying for proteins based on host response protein clusters and interactors. In one embodiment, the kit comprises various combinations of solid supports, such as a chip, a microtiter plate or a bead or resin and a plurality of capture reagents, e.g., biospecific capture reagents that bind to a plurality of different host response protein clusters. Thus, for example, the kits of the present invention can comprise mass spectrometry probes for SELDI, such as ProteinChip® arrays. In the case of biospecific capture reagents, the kit can comprise a solid support with a reactive surface, and a container comprising the biospecific capture reagent.

In one embodiment, this invention provides an array of biospecific capture reagents directed to a plurality of different host response protein clusters. The array can comprise a single solid support or a plurality of solid supports. The solid support of supports comprises a plurality of addressable locations. Each location comprises a biospecific capture reagent directed against a host response protein cluster. The array comprises a plurality of locations with different capture reagents arrayed so that different locations capture different host cell protein clusters. In particular, the locations can capture at least 2 different host response protein clusters, at least 4 different host response protein clusters, at least 8 different host response protein clusters, at least 16 different host response protein clusters, at least 24 different host response protein clusters, at least 48 different host response protein clusters, at least 96 different host response protein clusters, at least 384 different host response protein clusters or at least 1536 different host response protein clusters. More particularly, the array can comprise a plurality of locations each of which captures a different host response protein cluster selected from a different member of the class of host response proteins selected from the group consisting of positive acute phase reactants and negative acute phase reactants. Specific classes of positive acute phase reactants include most complement factors, most clotting factors, serum proteases and protease inhibitors, transport proteins such as haptoglobin and hemopexin, and inflammatory mediators such as serum amyloid A, c-reactive protein. Specific examples of negative acute phase reactants include albumin, transthyretin, transferring, fetuin, and insulin-like growth factor.

The array can comprise a biochip or collection of biochips to which the capture reagents are bound, or it could comprise a microtiter plate in which the capture reagents are bound to the surface of the wells of the microtiter, or it could comprise a microtiter plate comprising wells wherein each well comprises a chromatographic material derivatized with a biospecific capture reagent.

In another embodiment the kit of this invention comprises a plurality of biospecific capture reagents directed against a plurality of different host response protein clusters (and, preferably, against host response proteins of different classes) attached to at least one solid support. The solid support can be, for example, chromatographic material. In one embodiment, the kit comprises a plurality of packages, each of which contains a chromatographic material derivatized with a biospecific capture reagent directed against a host response protein cluster.

The kit can also comprise a washing solution or instructions for making a washing solution, in which the combination of the capture reagent and the washing solution allows capture of the protein or proteins on the solid support for subsequent detection by, e.g., mass spectrometry. The kit may include more than type of capture reagent, each present on a different solid support.

In a further embodiment, such a kit can comprise instructions for suitable operational parameters in the form of a label or separate insert. For example, the instructions may inform a consumer about how to collect the sample or how to wash the probe.

In yet another embodiment, the kit can comprise one or more containers with protein samples, to be used as standard(s) for calibration.

Having now generally described the invention, the same will be more readily understood through reference to the following exemplary embodiments, which are provided by way of illustration and are not intended to be limiting of the present invention unless specified.

Exemplary Embodiments

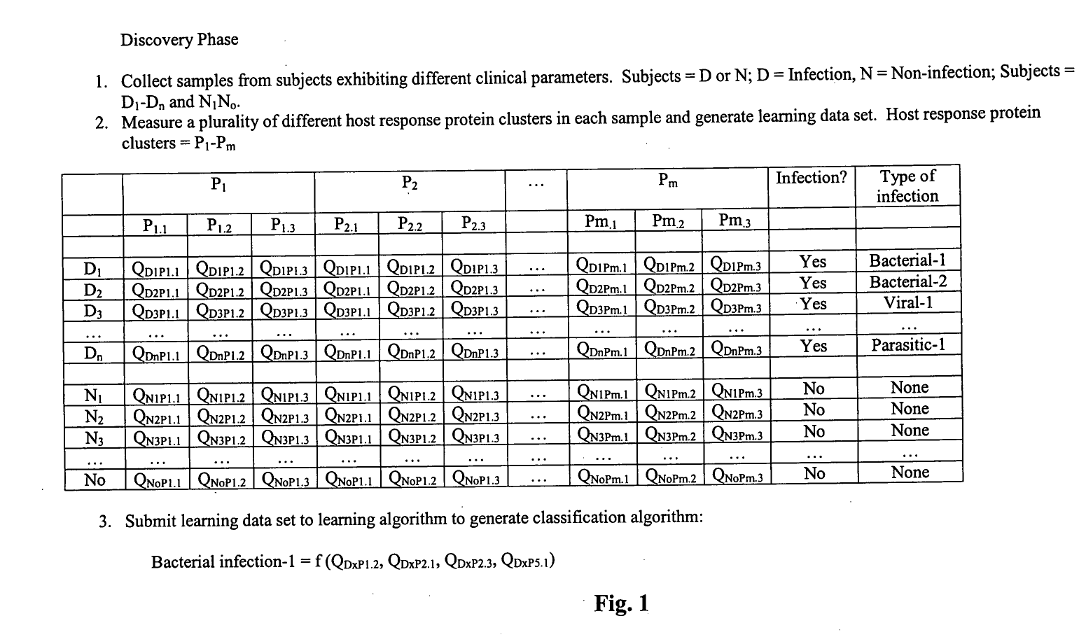

Referring to FIG. 1, the discovery phase involves the collection of samples from a statistically significant number of subjects falling into at least two groups exhibiting different clinical parameters. In this case, the subjects either exhibit infection (D) or non-infection (N). In the present example there are n subjects in class D and o subjects in class N. Those exhibiting infection may be further categorized as belong to different classes of infection, for example bacterial infection-1, bacterial infection-2, viral infection-1 and parasitic infection-1. (FIG. 1.1.)

In each sample a plurality of host response protein clusters are measured. The clusters are designated P₁, P₂. . . , Pₘ. For example the host response protein clusters might include C reactive protein (P₁), transthyretin (P₂), apolipoprotein A1 (P₃) inter-alpha trypsin inhibitor (P₄), albumin (P₅), . . . , and alpha-defensin (Pₘ). The members of the cluster can include the native protein, fragments of the native protein, and protein interactors (P_{1.1}, P_{1.2} and P_{1.3} (optionally to P_{1.p} depending on the number of cluster members captured)). Measurement involves, for example, capturing the proteins from the sample by binding them to a solid phase and removing un-bound proteins and then quantifying the amount captured by, for example, mass spectrometry. The amount of each protein in each host response protein cluster is quantified (e.g., by signal strength). In FIG. 1, the quantity of each protein is represented by Q_{D/NxPy.p}, in which Q is the quantity measured, D/Nₓ is the subject where D is diseased, N is non-diseased and x is a number from 1 to n or to o, and P_{y.q} is a host response protein in which y is a number from 1 to m representing a particular cluster and q is a number from 1 to p representing a particular protein within the cluster.

The measurements, Q_{D/NxPy.p}, are entered into a data base that identifies, for each subject, the amount of each protein detected in the various clusters. The identity of each sample, the amounts of protein measured and, usually, information about clinical parameters exhibited by the subject represent a data object. The collection of data objects for all the subjects represents a learning data set that can be subject to analysis by a learning algorithm. (FIG. 1.2.)

The learning algorithm selects particular proteins from the data set that, alone or together, are useful in a function for classifying a subject as belonging to class D or N, or to a particular disease sub-class. In this example, the classification algorithm found that bacterial infection-1 can be distinguished from non-bacterial infection by a function that includes measurements of Q_{DxP1.2}, Q_{DxP2.1}, Q_{DxP2.3}, Q_{DxP5.1}. Thus, bacterial infection-1=f (Q_{DxP1.2}, Q_{DxP2.1}, Q_{DxP2.3}, Q_{DxP5.1}), in which f is the function and Q_{DxP1.2}, Q_{DxP2.1}, Q_{DxP2.3}, Q_{DxP5.1} are the variables. (FIG. 1.3.)

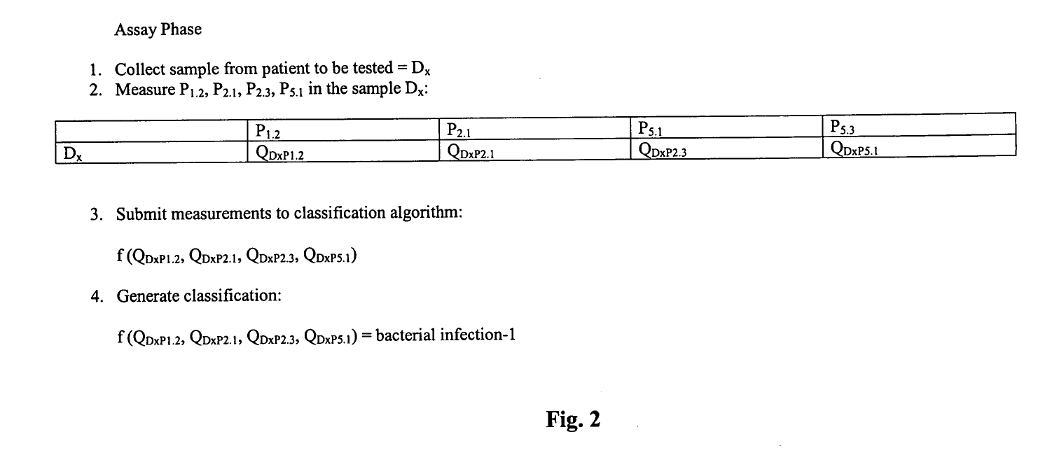

The classification algorithm is useful for performing a diagnostic test on an unknown subject, as shown in FIG. 2. A sample is collected from a subject, Dₓ. (FIG. 2.1.)

The proteins that are used in the diagnostic classification algorithm are then measured in the sample. In this case, this involves the measurement of P_{1.2}, P_{2.1}, P_{2.3}, and P_{5.1}. Thus, it is not necessary to measure any proteins in clusters P₃, P₄ or Pₘ. The measurements of particular proteins in clusters in P₁, P₂ and P₅ other than the ones used in the classification algorithm may be convenient, because they may be captured by antibodies used in the capture procedure, but is not necessary. (FIG. 2.2.)

The measurements, Q_{DxP1.2}, Q_{DXP2.1}, Q_{DXP2.3} and Q_{DXP5.1}, are submitted to the classification algorithm. (FIG. 2.3.) The classification algorithm performs the function on these quantities generating a result, which is the classification of the sample into a group. In this example, between the choices of bacterial infection-1 or not-bacterial infection-1, the classification algorithm assigned the sample D, to group bacterial infection-1. (FIG. 2.4.)

While specific examples have been provided, the above description is illustrative and not restrictive. Many variations of the invention will become apparent to those skilled in the art upon review of the specification. The scope of the invention should, therefore, be determined not with reference to the above description, but instead should be determined with reference to the appended claims along with their full scope of equivalents.

Although the foregoing invention has been described in detail by way of example for purposes of clarity of understanding, it will be apparent to the artisan that certain changes and modifications are comprehended by the disclosure and can be practiced without undue experimentation within the scope of the appended claims, which are presented by way of illustration not limitation.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

1. A method comprising:
a. collecting samples from subjects belonging to at least two groups that differ according to a clinical parameter associated with disease; and
b. measuring in each sample a plurality of host response protein clusters, wherein a cluster comprises a host response protein and at least one modified form of the host response protein;
c. submitting the measurements to a learning algorithm; and
d. generating a classification algorithm from the measurements that classifies a sample into at least one of the groups.

2. The method of claim 1 wherein the clinical parameter is selected from presence or absence of disease, risk of disease, the stage of disease, response to treatment of disease and disease prognosis.

3. The method of claim 1 wherein the disease is selected from an infectious disease, cancer, cardiovascular disease and autoimmune disease.

4. The method of claim 1 wherein the host response proteins are selected from C-reactive protein, transthyretin, apolipoprotein A1, apolipoprotein AII, apolipoprotein AIV, haptoglobin, interleukin 8, serum amyloid A (forms 1-4), inter-alpha trypsin inhibitor, complement factor, clotting cascade components, albumin, hemopexin, fetuin, transferrin, ceruloplasmin, serum proteases, and serum protease inhibitors and alpha-defensin.

5. The method of claim 1 wherein at least one modified form is selected from a splice variant, RNA editing, or a post-translational modification, e.g. a product of enzymatic degradation, glycosylation, phosphorylation, lipidation, oxidation, methylation, cystinylation, sulphonation and acetylation.

6. The method of claim 1, further comprising measuring at least one protein that interacts with a protein from at least one cluster.

7. The method of claim 1 wherein measuring comprises capturing each host response protein cluster with at least one biospecific capture reagent that specifically recognizes the host response protein and measuring the captured proteins.

8. The method of claim 1 wherein the host response protein clusters are measured by mass spectrometry.

9. The method of claim 1 wherein the host response protein clusters are measured by affinity mass spectrometry.

10. The method of claim 1 wherein the learning algorithm is selected from linear regression processes, binary decision trees, artificial neural networks such as back-propagation networks, discriminant analyses, logistic classifiers, and support vector classifiers.

11. The method of claim 1, further comprising using the classification algorithm to classify an unknown sample from a test subject into one of the groups.

12. A method comprising:
a. providing a learning set comprising a plurality of data objects representing subjects, wherein each data object comprises data representing measurements of a plurality of host response protein clusters from a subject sample, wherein each cluster comprises a host response protein and at least one modified form of the host response protein, and wherein the subjects are classified according to at least two different clinical parameters; and
b. training a learning algorithm with the learning set, thereby generating a classification model, wherein the classification model classifies a subject sample into a clinical parameter.

13. The method of claim 12 wherein the learning algorithm is selected from linear regression processes, binary decision trees, artificial neural networks, discriminant analyses, logistic classifiers, and support vector classifiers.

14. The method of claim 12 further comprising (1) submitting a data object to the classification algorithm for classification, wherein the data object represents a subject and comprises data representing measurements of proteins that are elements of the classification algorithm; and (2) using the classification algorithm to classify the subject.

15. A method comprising measuring in a sample a plurality of host response protein clusters, wherein a cluster comprises a host response protein and at least one modified form of the host response protein.

16. The method of claim 15 wherein measuring comprises capturing each host response protein cluster with at least one biospecific capture reagent that specifically recognizes the host response protein and measuring the captured proteins.

17. The method of claim 15 further comprising submitting the measurements to a learning algorithm.

18. A method comprising:
a. measuring a plurality of proteins in a sample, wherein the proteins are selected from host response proteins, modified forms of host response proteins and protein interactors with these, wherein the proteins are elements of a classification algorithm that classifies a sample into a group based on a clinical parameter, wherein the classification algorithm is generated according to the method of claim 12.

19. The method of claim 18 further comprising:
b. using the classification algorithm to classify the sample into a group based on the clinical parameter.

20. A kit comprising a plurality of biospecific capture reagents, wherein each capture reagent is attached to a different solid support or to a different addressable location on the same solid support or a combination of these, and wherein at least two of the capture reagents specifically bind to different host response protein clusters.

21. The kit of claim 20 wherein the solid support is a mass spectrometer probe.

22. A kit comprising a plurality of containers, each container comprising a different biospecific capture reagent, wherein each capture reagent specifically binds to a different host response protein cluster.

23. The kit of claim 22 further comprising at least one solid support comprising a reactive functionality for coupling a biospecific capture reagent to the solid support.

24. A method for measuring a clinical parameter in a subject comprising measuring in a sample from the subject a plurality of host response protein clusters, wherein a cluster comprises a host response protein and at least one modified form of the host response protein and correlating the measurement with a clinical parameter.

25. A method for assessing the presence or absence of a disease state in a subject comprising measuring in a sample from the subject a plurality of host response protein clusters, wherein a cluster comprises a host response protein and at least one modified form of the host response protein and correlating the measurement with the presence or absence of the disease state.

26. A method comprising:
a. collecting samples from subjects belonging to at least two groups that differ according to a clinical parameter associated with disease; and
b. measuring in each sample a plurality of host response proteins;
c. submitting the measurements to a learning algorithm; and
d. generating a classification algorithm from the measurements that classifies a sample into at least one of the groups.

27. A method comprising:
a. measuring a plurality of host response proteins in a sample, wherein the proteins are elements of a classification algorithm that classifies a sample into a group based on a clinical parameter; and
b. using the classification algorithm to classify the sample into a group characterized by clinical parameter.
